(19) United States
(12) Patent Application Publication
(10) Pub. No.: US 2005/0228054 A1
(43) Pub. Date: Oct. 13, 2005

(54) Methods for treating eye disorders

(76) Inventor: William G. Tatton, Fort MacLeod (CA)

Correspondence Address: LAHIVE & COCKFIELD, LLP., 28 STATE STREET, BOSTON, MA, 02109, US

(21) Appl. No.: 11/092,461

(22) Filed: Mar. 28, 2005

Related U.S. Application Data
(63) Continuation of application No. 10/700,604, filed on Oct. 3, 2003, now abandoned.
Application No. 10/700,604 is a continuation of application No. 10/349,150, filed on Jan. 21, 2003, now abandoned.
(60) Provisional application No. 60/349,948, filed on Jan. 18, 2002.

(51) Int. Cl. A61K 31/135

(52) U.S. Cl. 514/656; 607/86

(57) ABSTRACT

Methods for treating eye disorders by administering desmethyldeprenyl are described.

### RELATED APPLICATION

This application is a continuation of U.S. patent application Ser. No. 10/700,604, filed Nov. 3, 2003, entitled “Methods for Treating Eye Disorders,” which is a continuation of U.S. patent application Ser. No. 10/349,150, filed Jan. 21, 2003, entitled “Methods for Treating Eye Disorders,” which claims priority to U.S. Provisional Patent Application Ser. No. 60/349,948, entitled “Methods for Treating Eye Disorders,” filed Jan. 18, 2002. The entire contents of all of the above are hereby incorporated herein by reference.

### BACKGROUND OF THE INVENTION

Glaucoma is a disease of the eye characterized by elevated intraocular pressure. The elevated intraocular pressure leads to hardening of the eyeball, narrowing of the field of vision and a decrease in a subject's visual acuity. Glaucoma is a disease of the optic nerve and the elevated eye pressures are related to damage of this nerve. The optic nerve carries images from the retina to the brain. Glaucoma damages optical nerve cells causing blindspots to occur within a subject's vision. These blind spots typically are not noticed by the subject until considerable damage to the optic nerve has already occurred. The terminal stage of glaucoma is total blindness of the subject.

Approaches to treating glaucoma include the topical application of cholinergic agents, e.g., pilocarpine, alpha- or beta-adrenergic agonists or antagonists, e.g., clonidine, timolol or epinephrine. An alternative approach for treating glaucoma is the systemic administration of carbonic anhydrase inhibitors. In some cases laser or operative surgery is used to treat glaucoma.

Problems exist with the aforementioned approaches to treating glaucoma in that the treatments can be accompanied by side-effects. For example the instillation of a cholinergic agent, such as pilocarpine, into the eye of a subject can cause nausea diarrhea, muscular spasms, sweating, lacrimation, salivation, etc. Contraction of the pupil (myosis) and of the ciliary muscle of the eye, as well as dilation of the blood vessels of the iris and conjunctiva also can be observed. Visual complications, e.g., spasm of accommodation, myopia or a decrease in visual acuity, also can occur.

The treatment with a sympathomimetic agent such as dipivalylepinephrine is known frequently to produce sensations of burning or irritation in a subject. Another side-effect of these agents is the appearance of cardiac disturbances, e.g., palpitations, tachycardia, arrythmia, etc. Clonidine, which is known as an alpha-2-adrenergic receptor agonist, can bring about mydriasis, as well as an initial phase of ocular hypertension (biphasic effect). Furthermore, in spite of the topical application of the product to the eye, important systemic effects, such as bradycardia and hypotension, have been observed.

The use of beta-blocking medicaments also can cause important systemic effects after topical administration to the eye, due to the absence of a “first pass effect”. Timolol, for example, causes bradycardia or hypotension. These systemic secondary reactions to beta-blocking medicaments can reach such a severe level that the treatment has to be discontinued. Cases of suicidal depression, hallucinations, nightmares or psychoses requiring hospitalization have been reported in connection with these medicaments. Furthermore, these compounds have to be administered with extreme precautions to patients subject to cardiac or pulmonary functional disorders. In such patients, amongst others, cases of arrhythmia, cardiac arrest, asthma, dyspnea and bronchospasms have been reported.

The treatment with a sympatholytic agent, such as guanethidine, causes hyperemia of the conjunctiva and some irritation, not to mention the fact that these agents only have a low tendency to reduce intraocular pressure.

Finally, in the treatment of glaucoma with carbonic anhydrase inhibitors, such as acetazolamide or methazolamide, serious systemic side-effects, such as depression of the central nervous system, weight loss and, mainly, bone marrow hypofunction, have been reported.

The use of conventional hypotensive agents for the treatment of glaucoma is accompanied by considerable risks. Known medications are not particularly well suited for topical treatment and the systemic side-effects of these medicaments make them delicate to use because these effects are far from being negligible and because they can have, in some cases, severe consequences.

### SUMMARY OF THE INVENTION

In one embodiment, the invention pertains, at least in part, to methods for treating an eye disorder in a subject, by administering to the subject an effective amount of desmethyldeprenyl or a pharmaceutically acceptable salt thereof. In certain embodiments, desmethyldeprenyl may be administered in combination with other art recognized techniques for treating the eye disorder.

In another embodiment, the invention also pertains, at least in part, to pharmaceutical compositions, which contain an effective amount of desmethyldeprenyl or a pharmaceutically acceptable salt thereof, and a pharmaceutically acceptable carrier. Advantageously, the effective amount is effective to treat an eye disorder.

The invention also pertains, at least in part, to a packaged pharmaceutical composition. The packaged pharmaceutical composition includes an effective amount of desmethyldeprenyl, or a pharmaceutically acceptable salt thereof, and instructions for the use of desmethyldeprenyl for the treatment of an eye disorder.

In yet another embodiment, the invention pertains to a method for treating glaucoma in a subject, comprising administering to said subject an effective amount of desmethyldeprenyl.

In yet another embodiment, the invention pertains to a method for treating macular degeneration in a subject, comprising administering to said subject an effective amount of desmethyldeprenyl. In further embodiments, the macular degeneration may be exudative or non-exudative.

In yet another embodiment, the invention pertains to a method for treating retinitis pigmentosa in a subject, comprising administering to said subject an effective amount of desmethyldeprenyl.

In yet another embodiment, the invention pertains to a method for treating retinopathy in a subject, comprising administering to said subject an effective amount of desmethyldeprenyl. In a further embodiment, the retinopathy is diabetic retinopathy.

### DETAILED DESCRIPTION OF THE INVENTION

In one embodiment, the invention pertains, at least in part to a method for treating an eye disorder in a subject. The method includes administering to the subject an effective amount of desmethyldeprenyl or a pharmaceutically acceptable salt, or prodrug thereof.

The term “eye disorder” include disorders of the eye which can be treated by administration of desmethyldeprenyl. Examples of eye disorders include, but are not limited to, glaucoma, proliferative vitreoretinopathy (PVR), retinal detachment, corneopathies, non-exudative macular degeneration (dry AMD, e.g., age related), exudative (wet) AMD, retinopathies (e.g., diabetic), hereditary retinal degenerations including retinitis pigmentosa (hereditary and sporadic cases), Usher's syndrome, Fundus Albipunctatus, Stargardt's Disease, retinal degenerations owing to systemic inborn errors of metabolism (e.g., Tay-Sachs, Gauchers, Hereditary Telangiectasia), retrobulbar optic neuritis, Leber's congenital amaurosis, central or branch retinal artery occlusion, central or branch vein occlusion, photoreceptor degeneration (e.g., degeneration associated with chronic macular edema, toxic retinopathies due to systemic drugs, rhegmatogenous retinal detachment, non-rhegmatogenous retinal detachment, etc.), keratocyte loss (e.g., loss associated with excimer laser keratectomy such as Lasik and PRK), loss of conjunctival cells, loss of lacrimal gland cells (e.g., loss due to severe allergic reactions such as Stevens Johnson syndrome, Sjogren's Syndrome, keratoconjunctivitis sicca, radiation therapy, etc.), loss of motor nerve function in diabetic and non-diabetic oculomotor nerve palsies, loss of visual field (e.g., loss due to ischemia, tumor pressure, and radiation-induced damage of the visual cortex of the occipital lobe, the optic radiation, the lateral geniculate, the optic tracts, chiasm, and/or optic nerve), and other diseases or disorders of the eye associated with apoptosis.

The term “desmethyldeprenyl” includes compounds of the formula: 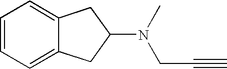
and pharmaceutically acceptable salts and prodrugs thereof. Desmethyldeprenyl used in the methods of the invention may include (−) isomer of desmethyldeprenyl, the (+) isomer of desmethyldeprenyl, or mixtures thereof. The invention, also pertains, at least in part, to methods of treating eye disorders, as described herein, with the deprenyl compounds described in, for example, U.S. patent application Nos. 08/470,301, U.S. Ser. No. 08/374,332; U.S. Pat. No. 5,444,095; and U.S. Pat. No. 5,981,598. It also pertains to pharmaceutical compositions and packaged pharmaceutical compositions comprising the deprenyl compounds.

The term “prodrug” includes moieties which may be cleaved in vivo, to yield an active compound. The prodrug may be metabolized in vivo by enzymes or by other mechanisms to desmethyldeprenyl. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the desmethyldeprenyl, or by separately reacting desmethyldeprenyl with a suitable derivatizing agent.

The term “subject” includes organisms capable of suffering from an eye disorder, such as mammals (e.g. primates (e.g., monkeys, gorillas, chimpanzees, and, advantageously, humans), goats, cattle, horses, sheep, dogs, cats, mice, rats, rabbits, pigs, dolphins, ferrets, squirrels), reptiles, or fish, and transgenic species thereof. In one embodiment, the subject is suffering from or at risk of suffering from an eye disorder. The term subject is intended to include living organisms in which eye disorders can occur, e.g., mammals.

The term “therapeutically effective amount” or “effective amount” includes an amount of the compound which is effective in treating an eye disorder. A therapeutically effective amount may be readily determined by an attending diagnostician, as one skilled in the art, by the use of known techniques and by observing results obtained under analogous circumstances.

The term “treated,” “treating” or “treatment” includes the diminishment or alleviation of at least one symptom associated or caused by eye disorder being treated. For example, treatment can be diminishment of one or several symptoms of a disorder or complete eradication of a disorder.

The term “pharmaceutically acceptable salt” includes relatively non-toxic, inorganic and organic acid addition salts of desmethyldeprenyl. These salts can be prepared in situ during the final isolation and purification of desmethyldeprenyl, or by separately reacting a purified desmethyldeprenyl in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

In a further embodiment, desmethyldeprenyl is administered in combination with a known method of treating the eye disorder.

The term “in combination with” a known method of treatment is intended to include simultaneous administration of or treatment with desmethyldeprenyl and the known method of treatment, administration of or treatment with desmethyldeprenyl first, followed by the known method of treatment and administration of or treatment with the known method of treatment first, followed by desmethyldeprenyl second. Any of the therapeutically useful method known in the art for treating a particular eye disorder can be used in the methods of the invention.

Examples of known methods of treating eye disorders include, but are not limited to the methods described.

Known methods for treatment of non-exudative age-related macular degeneration (dry AMD) include the administration of luten and sub-acute diode laser treatment.

Known methods of treatment of exudative (wet) AMD include laser photocoagulation and photodynamic therapy.

Known methods of treating retinopathies, such as, for example diabetic retinopathy, include oral hypoglycemics and laser treatments (e.g., focal and pan-retinal laser photocoagulation).

Examples of treatments for hereditary retinal degeneration, such as retinitis pigmentosa (e.g., both hereditary and sporadic cases), Usher's syndrome, Fundus Albipunctatus, and Stargardt's Disease include administering Vitamin A supplements, and potentially, gene therapies in the future.

Known methods of treatment of field loss, e.g., field loss due to glaucoma, include, but are not limited to trabeculoplasty, iridectomy, iridotomy, filtration surgery, administration of drugs that increase aqueous outflow through the trabecular meshwork or through the uveal tract, and administration of drugs that decrease aqueous production.

Examples of known methods of treatment for retrobulbar optic neuritis include the administration of steroids.

Known methods for treating central or branch retinal artery occlusions include the administration of anticoagulants and clot busting drugs as well as laser treatments. Central or branch vein occlusions may be treated using similar methods.

Photoreceptor degeneration, such as that associated with chronic macular edema, is generally treated by the administration of steroids. For the treatment of toxic retinopathies due to systemic drugs, a known method of treatment includes withdrawal of the drug.

Examples of known methods of treating photoreceptor degeneration associated with rhegmatogenous retinal detachment, include repairing the detachment.

Known methods for treating photoreceptor degeneration associated with non-rhegmatogenous retinal detachment, include eliminating the cause of the exudative detachment (e.g., by a subretinal neurovascular net).

Methods of treating a loss of conjunctival cells or a loss of lacrimal gland cells in severe allergic reactions (e.g., Stevens Johnson syndrome) include withdrawing the drug causing the allergic reaction or by administering steroids.

Known methods of treating a loss of visual field owing to ischemia, tumor pressure, or radiation-induced damage of the visual cortex of the occipital lobe, the optic radiation, the lateral geniculate, the optic tracts, chiasm, or the optic nerve, include the administration of steroids or clot busting drugs, and, when appropriate, removing tumors.

The invention also pertains, at least in part, to a pharmaceutical composition comprising an effective amount of desmethyldeprenyl or a pharmaceutically acceptable salt thereof, and a pharmaceutically acceptable carrier. Advantageously, the effective amount is effective to treat an eye disorder, such as, for example, non-exudative age-related macular degeneration, exudative age-related macular degeneration, retinopathy, retinal degeneration, retinitis pigmentosa, Usher's syndrome, fundus albipunctatus, Stargardt's disease, Tay-Sachs, Gauchers, hereditary telangiectasia, glaucoma, retrobulbar optic neuritis, Leber's congenital amaurosis, central or branch retinal artery occlusion, central or branch vein occlusion, photoreceptor degeneration, keratocyte loss, loss of conjunctival cells, lacrimal gland cells, Stevens Johnson syndrome, Sjogren's Syndrome, keratoconjunctivitis sicca, loss of motor nerve function, or loss of visual field.

Desmethyldeprenyl can be incorporated into pharmaceutical compositions suitable for administration. Such compositions typically comprise desmethyldeprenyl and a pharmaceutically acceptable carrier. The language “pharmaceutically acceptable carrier” includes any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g. desmethyldeprenyl) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, desmethyldeprenyl may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, desmethyldeprenyl is formulated into ointments, salves, gels, or creams as generally known in the art.

Desmethyldeprenyl can also be prepared in the form of suppositories (e.g., with conventional suppository bases such as cocoa butter and other glycerides) or retention enemas for rectal delivery.

In one embodiment, desmethyldeprenyl is prepared with carriers that will protect it against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc.

Liposomal suspensions (including liposomes targeted to specific cells with antibodies to specific antigens) can also be used as pharmaceutically acceptable carriers. These can be prepared according to methods known to those skilled in the art, for example, as described in U.S. Pat. No. 4,522,811. Lipid based delivery systems have the advantage of being able to deliver hydrophobic drugs. Another delivery system for hydrophobic drugs is a cochleate delivery system from BioDelivery Sciences International, as described in U.S. Pat. No. 6,153,217.

Referred to as the PHOTOTARGET® system, light-targeted delivery of drugs and/or diagnostic imaging dyes to the vasculature of the retina is a potential delivery mechanism for desmethyldeprenyl. The method includes intravenous administration of a liposome vesicles which comprise artificial phospholipids encapsulating desmethyldeprenyl. A short, low-intensity pulse of light delivered warming of the target tissue (retinal or choroidal blood vessels) thereby thermally rupturing the liposomes and releasing a small bolus of desmethyldeprenyl from circulating liposomes. The intensity of the light alone is insufficient to damage either the targeted or the surrounding tissues (See, for example, U.S. Pat. No. 6,248,727; U.S. Pat. No. 6,140,314; U.S. Pat. No. 5,935,942; U.S. Pat. No. 4,891,043).

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of desmethyldeprenyl calculated to produce the desired therapeutic effect in association with the required pharmaceutical carrier. The specification for the dosage unit forms of the invention are dictated by and directly dependent on the unique characteristics of desmethyldeprenyl and the particular therapeutic effect to be achieved, and the limitations inherent in the art of compounding desmethyldeprenyl for the treatment of individuals.

Toxicity and therapeutic efficacy of desmethyldeprenyl can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of desmethyldeprenyl lies preferably within a range of circulating concentrations that include the ED50 with little or no toxicity. The dosage may vary within this range depending upon the dosage form employed and the route of administration utilized. For desmethyldeprenyl, the therapeutically effective dose can be estimated initially from cell culture assays. A dose can be formulated in animal models to achieve a circulating plasma concentration range that includes the IC₅₀ (i.e., the concentration of desmethylprenyl which achieves a half-maximal inhibition of symptoms) as determined in cell culture. Such information can be used to more accurately determine useful doses in humans. Levels in plasma can be measured, for example, by high performance liquid chromatography.

Desmethyldeprenyl is administered to subjects in a biologically compatible form suitable for pharmaceutical administration in vivo to treat eye disorders. By “biologically compatible form suitable for administration in vivo” is meant a form of the molecule to be administered in which any toxic effects are outweighed by the therapeutic effects of the protein. Administration of desmethyldeprenyl as described herein can be in any pharmacological form including a therapeutically active amount of desmethyldeprenyl alone or in combination with a pharmaceutically acceptable carrier.

The invention also pertains, at least in part to a packaged pharmaceutical composition, including desmethyldeprenyl, or a pharmaceutically acceptable salt thereof, and instructions for the use of desmethyldeprenyl for the treatment of an eye disorder. The packaged pharmaceutical composition may also include a container and a pharmaceutically acceptable carrier. In a durther embodiment, the desmethyldeprenyl is packaged in an effective amount, e.g., an amount effective to treat an eye disorder. The pharmaceutical compositions can be included in a container, pack, or dispenser together with instructions for administration, e.g., to treat an eye disorder.

In a further embodiment, the invention pertains to a method for treating glaucoma in a subject, by administering an effective amount of desmethyldeprenyl to the subject.

In yet another embodiment, the invention pertains to a method for treating macular degeneration in a subject, comprising administering to said subject an effective amount of desmethyldeprenyl. In further embodiments, the macular degeneration may be exudative or non-exudative.

In yet another embodiment, the invention pertains to a method for treating retinitis pigmentosa in a subject, comprising administering to said subject an effective amount of desmethyldeprenyl.

In yet another embodiment, the invention pertains to a method for treating retinopathy in a subject, comprising administering to said subject an effective amount of desmethyldeprenyl. In a further embodiment, the retinopathy is diabetic retinopathy.

### Equivalents

Those skilled in the art will recognize, or be able to ascertain using no more than routine experimentation, many equivalents to the specific embodiments and methods described herein. Such equivalents are intended to be encompassed by the scope of the following claims.

All patents, patent applications, and literature references cited herein are hereby expressly incorporated by reference.

1. A method for treating an eye disorder in a subject, comprising administering to said subject an effective amount of desmethyldeprenyl or a pharmaceutically acceptable salt thereof, such that said eye disorder is treated.

2. The method of claim 1, further comprising administering desmethyldeprenyl in combination with a supplementary method for treating said eye disorder.

3. The method of claim 1, wherein said eye disorder is glaucoma.

4. The method of claim 3, wherein said supplementary method is trabeculoplasty, iridectomy, iridotomy, filtration surgery, or known glaucoma treatment agents.

5. The method of claim 1, wherein said eye disorder is non-exudative age-related inacular degeneration.

6. The method of claim 5, wherein said supplementary method is luten or sub-acute diode laser treatment.

7. The method of claim 1, wherein said eye disorder is exudative age-related macular degeneration.

8. The method of claim 7, wherein said supplementary method is laser photocoagulation or photodynamic therapy.

9. The method of claim 1, wherein said eye disorder is retinopathy.

10. The method of claim 9, wherein said supplementary method is administering hypoglycemics, laser treatment or withdrawing toxic drugs.

11. The method of claim 1, wherein said eye disorder is retinitis pigmentosa, Usher's syndrome, fundus albipunctatus, or Stargardt's disease.

12. The method of claim 11, wherein said supplementary method is administering vitamin A or nucleic acids.

13. The method of claim 1, wherein said eye disorder is Tay-Sachs, keratocyte loss, lacrimal gland cell loss, oculomotor nerve palsy, Gauchers, Leber's Congenital Amaurosis or hereditary telangiectasia.

14. The method of claim 1, wherein said eye disorder is retrobulbar optic neuritis or photoreceptor degeneration.

15. The method of claim 14, wherein said supplementary method is administering steroids.

16. The method of claim 1, wherein said eye disorder is a central or branch retinal artery or vein occlusion.

17. The method of claim 16, wherein said supplementary method is laser treatment or the administering anticoagulants or clot busters.

18. The method of claim 1, wherein said eye disorder is photoreceptor degeneration.

19. The method of claim 18, wherein said supplementary treatment is repairing the detachment or treating the cause of the detachment.

20. The method of claim 1, wherein said eye disorder is loss of conjunctival cells or lacrimal gland cells.

21. The method of claim 20, wherein said supplementary treatment comprises administering steroids.

22. The method of claim 1, wherein said eye disorder is loss of visual field.

23. The method of claim 22, wherein said supplementary treatment comprises administration of steroids or clot busting drugs.

24. The method of claim 1, wherein said subject is a mammal.

25. The method of claim 24, wherein said mammal is a human.

26. The method of claim 25, wherein said human is suffering from or at risk of suffering from an eye disorder.

27. The method of claim 1, further comprising administering said desmethyldeprenyl in combination with a pharmaceutically acceptable carrier.

28. A pharmaceutical composition comprising an effective amount of desmethyldeprenyl or a pharmaceutically acceptable salt thereof, and a pharmaceutically acceptable carrier.

29. The pharmaceutical composition of claim 28, wherein said effective amount is effective to treat an eye disorder.

30. The pharmaceutical composition of claim 29, wherein said eye disorder is non-exudative age-related macular degeneration, exudative age-related macular degeneration, retinopathy, retinal degeneration, retinitis pigmentosa, Usher's syndrome, fundus albipunctatus, Stargardt's disease, Tay-Sachs, Gauchers, hereditary telangiectasia, glaucoma, retrobulbar optic neuritis, Leber's congenital amaurosis, central or branch retinal artery occlusion, central or branch vein occlusion, photoreceptor degeneration, keratocyte loss, loss of conjunctival cells, lacrimal gland cells, Stevens Johnson syndrome, Sjogren's Syndrome, keratoconjunctivitis sicca, loss of motor nerve finction, or loss of visual field.

31. A packaged pharmaceutical composition comprising:
an effective amount of desmethyldeprenyl, or a pharmaceutically acceptable salt thereof, and
instructions for the use of desmethyldeprenyl for the treatment of an eye disorder.

32. The packaged pharmaceutical composition of claim 31, wherein said effective amount is effective to treat said eye disorder.

33. The pharmaceutical composition of claim 31, wherein said eye disorder is non-exudative macular degeneration, exudative macular degeneration, retinopathy, retinal degeneration, retinitis pigmentosa, Usher's syndrome, fundus albipunctatus, Stargardt's disease, Tay-Sachs, Gauchers, hereditary telangiectasia, glaucoma, retrobulbar optic neuritis, Leber's congenital amaurosis, central or branch retinal artery occlusion, central or branch vein occlusion, photoreceptor degeneration, keratocyte loss, loss of conjunctival cells, lacrimal gland cells, Stevens Johnson syndrome, Sjogren's Syndrome, keratoconjunctivitis sicca, loss of motor nerve function, or loss of visual field.

34. A method for treating glaucoma in a subject, comprising administering to said subject an effective amount of desmethyldeprenyl or a pharmaceutically acceptable salt thereof, such said subject is treated for glaucoma.

35. A method for treating macular degeneration in a subject, comprising administering to said subject an effective amount of desmethyldeprenyl or a pharmaceutically acceptable salt thereof, such that said subject is treated for macular degeneration.

36. The method of claim 35, wherein said macular degeneration is exudative macular degeneration.

37. The method of claim 35, wherein said macular degeneration is non-exudative macular degeneration.

38. A method for treating retinitis pigmentosa in a subject, comprising administering to said subject an effective amount of desmethyldeprenyl or a pharmaceutically acceptable salt thereof, such that said subject is treated for retinitis pigmentosa.

39. A method for treating retinopathy in a subject, comprising administering to said subject an effective amount of desmethyldeprenyl or a pharmaceutically acceptable salt thereof, such that said subject is treated for retinopathy.

40. The method of claim 39, wherein said retinopathy is diabetic retinopathy.

41. The method of any one of claims 34, 35, 38, or 39, wherein said subject is a human.

42. The method of any one of claims 34, 35, 38, or 39, further comprising administering said desmethyldeprenyl in combination with a pharmaceutically acceptable carrier.
